(19) United States
(12) Patent Application Publication
(10) Pub. No.: US 2008/0139480 A1
(43) Pub. Date: Jun. 12, 2008

(54) Combination Of (A) N--4-(3- Pyridyl)-2-Pyrimidine-Amine And (B) At Least One Hypusination Inhibitor And The UseThereof

(76) Inventors: Tim H. Bruemmendorf, Hamburg (DE); Stefan Balabanov, Hamburg (DE); Ulrike Hartmann, Tubingen (DE); Winfried Kammer, Rottenburg-Kiebingen (DE); Alfred Nordheim, Dettenhausen (DE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/583,107

(22) PCT Filed: Dec. 17, 2004
(86) PCT No.: PCT/EP2004/014439

§ 371 Date: Jul. 17, 2007

Related U.S. Application Data
(60) Provisional application No. 60/531,563, filed on Dec. 19, 2003.

(51) Int. Cl. A61K 38/08; A61K 31/506; A61P 35/00; A61P 35/02

(52) U.S. Cl. 514/17; 514/275

(57) ABSTRACT

The invention relates to a method of treating a warm-blooded animal, especially a human, having a proliferative disease comprising administering to the animal a combination which comprises (a) N-{5-[4-(4-methyl-piperazino-methyl)-benzoylamido]-2-methylphenyl}-4-(3-pyridyl)-2-pyrimidine-amine and (b) at least one hypusination inhibitor, to a combination comprising (a) and (b) as defined above and optionally at least one pharmaceutically acceptable carrier for simultaneous, separate or sequential use, in particular for the delay of progression or treatment of a proliferative disease and finally to the use of at least one hypusination inhibitor for the preparation of a medicament for the delay of progression or treatment of leukemia, particularly Imatinib-resistant leukemia.

The invention relates to a method of treating a warm-blooded animal, especially a human, having a proliferative disease comprising administering to the animal a combination which comprises (a) N-{5-[4-(4-methyl-piperazino-methyl)-benzoylamido]-2-methylphenyl}-4-(3-pyridyl)-2-pyrimidine-amine and (b) at least one hypusination inhibitor, especially as defined herein; a combination comprising (a) and (b) as defined above and optionally at least one pharmaceutically acceptable carrier for simultaneous, separate or sequential use, in particular for the delay of progression or treatment of a proliferative disease, especially a tumor disease or leukemia; a pharmaceutical composition comprising such a combination; the use of such a combination for the preparation of a medicament for the delay of progression or treatment of a proliferative disease, and finally to the use of at least one hypusination inhibitor for the preparation of a medicament for the delay of progression or treatment of an Imatinib-resistant leukemia; and to a commercial package or product comprising such a combination.

The preparation of N-{5-[4-(4-methyl-piperazino-methyl)-benzoylamido]-2-methylphenyl}-4-(3-pyridyl)-2-pyrimidine-amine and the use thereof, especially as an antiproliferative agent, are described in EP-A-0 564 409, which was published on 6 Oct. 1993, and in equivalent applications in numerous other countries, e.g., U.S. Pat. No. 5,521,184. This compound is also known and hereinafter referred to as Imatinib [International Non-proprietary Name].

The selective tyrosine kinase inhibitor Imatinib (formerly ST1571, Gleevec®) has been shown to block phosphorylation of tyrosine residues by occupying the ATP binding site of the Abl tyrosine kinases Bcr-Abl, c-Abl, v-Abl and Abl-related gene (ARG) as well as platelet-derived growth factor receptors (PDGF) alpha and beta and the receptor for human stem cell factor (SCF) c-kit. Based on numerous studies with chronic myeloid leukemia (CML), including studies with patients in early chronic phase (CP), accelerated phase (AP) and myeloid blast crisis (BC), Imatinib is considered the new gold standard of treatment for chronic myeloid leukemia. Additionally, Imatinib induces sustained responses in individuals with gastro-intestinal stromal tumors (GIST), a tumor entity with constitutive activation of c-kit and in patients with myeloproliferative diseases and rearrangements in the PDGF-R-beta gene on chromosome 5q33.

Despite these promising results, particularly in CP, the development of resistance to Imatinib occurs frequently in AP and BC and remissions usually only lasts for 6-12 months. Therefore, particularly for late stage disease, synergistic treatment strategies are urgently warranted.

To further enhance therapeutic success, novel screening strategies for syngergistic treatment approaches need to be established. As disclosed herein, differential protein expression analysis of Imatinib-treated as opposed to untreated Bcr-Abl positive cell lines are used in order to detect proteins that are regulated by Bcr-Abl expression and that could potentially serve as novel targets for synergistic therapeutic intervention.

Surprisingly, it has now been found that cellular cytotoxicity and apoptosis in a Bcr-Abl cell line in the presence of a combination which comprises (a) Imatinib or pharmaceutically acceptable salts thereof, and (b) at least one hypusination inhibitor is greater than the effects that can be achieved with either type of combination partner alone, i.e. a supra-additive or synergistic effect. Thus, it is contemplated herein that this combination may be used to treat a proliferative disease, particularly for treating leukemia including, but not limited to, Imatinib-resistant leukemia. It is further contemplated that hypusination inhibitors are particularly useful for treating leukemia, particularly leukemia resistant to Imatinib or pharmaceutically acceptable salts thereof.

Hence, in a first embodiment, the present invention relates to a method of treating a warm-blooded animal having leukemia, particularly Imatinib-resistant leukemia, comprising administering to the animal at least one hypusination inhibitor in a quantity which is therapeutically effective against leukemia, in which method said compounds can also be present in the form of their pharmaceutically acceptable salts.

In a second embodiment, the present invention relates to the use of at least one hypusination inhibitor for the manufacture of a drug useful for treating a warm-blooded animal having leukemia, particularly Imatinib-resistant leukemia.

In a third embodiment, the present invention relates to a method of treating a warm-blooded animal having leukemia, particularly Imatinib-resistant leukemia, comprising administering to the animal at least one hypusination inhibitor in a quantity which is therapeutically effective against leukemia, in which method said compounds can also be present in the form of their pharmaceutically acceptable salts.

Furthermore, the present invention relates to a combination, such as a combined preparation or a pharmaceutical composition, which comprises (a) N-{5-[4-(4-methyl-piperazino-methyl)-benzoylamido]-2-methylphenyl}-4-(3-pyridyl)-2-pyrimidine-amine and (b) at least one hypusination inhibitor, wherein the active ingredients are present in each case in free form or in the form of a pharmaceutically acceptable salt, and optionally at least one pharmaceutically acceptable carrier; for simultaneous, separate or sequential use.

The present invention also concerns a method of treating a warm-blooded animal having a proliferative disease comprising administering to the animal a combination which comprises (a) N-{5-[4-(4-methyl-piperazino-methyl)-benzoylamido]-2-methylphenyl}-4-(3-pyridyl)-2-pyrimidine-amine and (b) at least one hypusination inhibitor, in a quantity which is jointly therapeutically effective against a proliferative disease and in which the compounds can also be present in the form of their pharmaceutically acceptable salts.

Furthermore, the present invention pertains to a pharmaceutical composition comprising a quantity of a combination as defined herein and at least one pharmaceutically acceptable carrier which is jointly therapeutically effective against a proliferative disease.

In the herein disclosed methods, combinations, compositions or uses, the combination partners (a) and (b) are preferably administered in synergistically effective amounts.

The term “proliferative disease” includes malignant and non-malignant proliferative diseases, e.g. atherosclerosis, carcinomas and leukemia, tumors, thrombosis, psoriasis, restenosis, sclerodermitis and fibrosis.

The term “tumor” as used herein includes, but is not limited to breast cancer, melanoma, epidermoid cancer, cancer of the colon and generally the GI tract, GIST, lung cancer, in particular small-cell lung cancer, and non-small-cell lung cancer, head and neck cancer, genitourinary cancer, e.g. cervical, uterine, ovarian, testicles, prostate or bladder cancer, Hodgkin's disease or Kaposi's sarcoma. It is contemplated that the combinations of the present invention are useful to inhibit the growth of liquid tumors and, in particular, solid tumors. Furthermore, depending on the tumor type and the particular combination used a decrease of the tumor volume may be obtained. The combinations disclosed herein are also suited to prevent the metastatic spread of tumors and the growth or development of micrometastases. The combinations disclosed herein are in particular suitable for the treatment of poor prognosis patients, e.g. such poor prognosis patients having non-small-cell lung cancer or Imatinib-resistant leukemia.

The term “leukemia” as used herein includes, but is not limited to, chronic myelogenous leukemia (CML) and acute lymphocyte leukemia (ALL), especially Philadelphia-chromosome positive acute lymphocyte leukemia (Ph+ ALL) as well as Imatinib-resistant leukemia. Preferably, the variant of leukemia to be treated by the methods disclosed herein is CML.

The term “Imatinib-resistant leukemia” as used herein defines especially a leukemia for which Imatinib is no longer therapeutically efficient or has a reduced therapeutic effectiveness.

The term “a combined preparation”, as used herein defines especially a “kit of parts” in the sense that the combination partners (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b). The ratio of the total amounts of the combination partner (a) to the combination partner (b) to be administered in the combined preparation can be varied, e.g. in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners (a) and (b), in particular a synergism, e.g. a more than additive effect, additional advantageous effects, less side effects, a combined therapeutic effect in a non-effective dosage of one or both of the combination partners (a) and (b), and very preferably a strong synergism of the combination partners (a) and (b).

The term “delay of progression” as used herein means administration of the combination to patients being in a pre-stage or in an early phase of the disease to be treated, in which patients for example a pre-form of the corresponding disease is diagnosed or which patients are in a condition, e.g. during a medical treatment or a condition resulting from an accident, under which it is likely that a corresponding disease will develop.

It will be understood that references to the combination partners (a) and (b) are meant to also include the pharmaceutically acceptable salts. If this combination partners (a) and (b) have, for example, at least one basic center, they can form acid addition salts. Corresponding acid addition salts can also be formed having, if desired, an additionally present basic center. The combination partners (a) and (b) having an acid group (for example COOH) can also form salts with bases. The combination partner (a) or (b) or a pharmaceutically acceptable salt thereof may also be used in form of a hydrate or include other solvents used for crystallization. N-{5-[4-(4-methyl-piperazino-methyl)-benzoylamido]-2-methylphenyl}-4-(3-pyridyl)-2-pyrimidine-amine, i.e. combination partner (a), is preferably used in the present invention in the form of its monomesylate salt. Depending on the chemical structure of the hypusination inhibitor, a salt form thereof may not exist.

The combination partner (a) can be prepared and administered as described in WO 99/03854, especially the monomesylate salt of N-{5-[4-(4-methyl-piperazino-methyl)-benzoylamido]-2-methylphenyl}-4-(3-pyridyl)-2-pyrimidine-amine can be formulated as described in Examples 4 and 6 of WO 99/03854. The drug can be applied, e.g., in the form of a pharmaceutical composition as disclosed in WO03/090720.

The term “hypusination inhibitor” defines a reagent, drug or chemical which is able to decrease the formation of hypusine in vitro or in vivo. Hypusine is a unique amino acid formed by a posttranslational modification of a lysine residue in eukaryotic initiation faction 5A (eIF-5A) and is a critical for cell survival and proliferation (see, for example, Chen et al., J. Chin. Chem. Soc., Vol. 46, No. 5, 1999). Recently, data indicate that cell proliferation may be inhibited in cells in vitro by exposure to chelating molecules such as ciclopirox, deferiprone and deferoxamine which target deoxyhypusine hydroxylase, a metalloenzyme necessary for hypusine formation (Clement, et al. Int. J. Cancer 2002 Aug. 1; 100(4):491-8).

Hypusination inhibitors can be readily identified using standard screening protocols in which a cellular extract or other preparation possessing conditions suitable for hypusine formation is placed in contact with a potential inhibitor, and the level of hypusination activity measured in the presence or absence of the inhibitor, or in the presence of varying amounts of inhibitor. In this way, not only can useful inhibitors be identified, but the optimum level of such an inhibitor can be determined in vitro for further testing in vivo. Examples of suitable hypusination inhibitors are familiar to one of skill in the art and include, but are not limited to, ciclopirox, deoxyspergualin, interferon alpha, deferoxamine, deferiprone as well as additional compounds belonging to the family of hydroxypyridones (see, e.g., Mycoses 1997; 40:243-247) as well as other compounds with activity as iron chelators. The latter includes compounds disclosed in PCT publications WO 03/039541, WO 97/49395 and U.S. Pat. Nos. 6,465,504 and 6,596,750, e.g., substituted 3,5-diphenyl-1,2,4-triazoles.

Suitable hypusination inhibitors also include, but are not limited to, those that inhibit hypusination by inhibiting the activity of enzymes necessary for hypusine formation, e.g., deoxyspergualin and N¹-guanyl-1,7-diaminoheptane (GC-7; see e.g., Jansson et al., J. Bacteriology 182: No. 4, 1158-1161) which act by blocking deoxyhypusine-synthase, and ciclopirox and deferoxamine which inhibit deoxyhypusine-hydroxylase.

In a preferred embodiment of the invention the hypusination inhibitor is ciclopirox or 6-cyclohexyl-1-hydroxy-4-methyl-2(1H)-pyridinone, a common antifungal drug well known to one of skill in the art (see, for example, Gupta A. K. and Skiner A. R., Intl. J. Dermatol. 2003 Sept; 42 Suppl 1:3-9) and widely commercially available (e.g. Sigma, Taufkirchen, Germany).

In another preferred embodiment, the hypusination inhibitor is 4-[3,5-bis(2-hydroxyphenyl)-[1,2,4]triazol-1-yl]benzoic acid. The compound and its preparation is described, e.g., in U.S. Pat. No. 6,465,504 B1. The drug can be applied, e.g., as described in U.S. Pat. No. 6,465,504 B1 or WO2004/035026.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

A combination which comprises (a) N-{5-[4-(4-methyl-piperazino-methyl)-benzoylamido]-2-methylphenyl}-4-(3-pyridyl)-2-pyrimidine-amine and (b) at least one hypusination inhibitor, in which the active ingredients are present in each case in free form or in the form of a pharmaceutically acceptable salt and optionally at least one pharmaceutically acceptable carrier, will be referred to hereinafter as a COMBINATION OF THE INVENTION. Depending on the structure of the hypusination inhibitor, a salt form may be impossible.

The nature of proliferative diseases like solid tumor diseases is multifactorial. Under certain circumstances, drugs with different mechanisms of action may be combined. However, just considering any combination of drugs having different mode of action does not necessarily lead to combinations with advantageous effects.

The utility of the invention for the treatment of proliferative diseases such as leukemia is demonstrated, by the ability of the COMBINATION OF THE INVENTION to act synergistically to cause cellular cytotoxicity and induce apoptosis. Specifically, while data indicates that as a single agent ciclopirox inhibits cell viability and induces apoptosis of K562 and HL-60 cells, the combination of ciclopirox and Imatinib shows a synergistic effect on both cellular cytotoxicity and induction of apoptosis in these cells. No such synergistic effect is observed in Bcr-Abl-negative HL-60 control cells. Based on these data and since a number of hypusination inhibitors are clinically approved drugs with acceptable toxicity profiles, our results have important implications for the design of novel synergistic treatment strategies for patients with Bcr-Abl-positive leukemias and potentially for other Imatinib-responsive diseases.

A further benefit is that lower doses of the active ingredients of the COMBINATION OF THE INVENTION can be used, for example, that the dosages need not only often be smaller but are also applied less frequently, or can be used in order to diminish the incidence of side-effects. This is in accordance with the desires and requirements of the patients to be treated. This supra-additive interaction is not associated with a similar increase in adverse effects potential.

It can be shown by established test models and in particular those test models described herein that a COMBINATION OF THE INVENTION may be used in a more effective delay of progression or treatment of a proliferative disease compared to the effects observed with the single combination partners. The person skilled in the pertinent art is fully enabled to select a relevant test model to prove the hereinbefore and hereinafter mentioned therapeutic indications and beneficial effects. The pharmacological activity of a COMBINATION OF THE INVENTION may, for example, be demonstrated in a clinical study or in a test procedure as essentially described hereinafter.

Suitable clinical studies are, for example, open label non-randomized, dose escalation studies in patients with advanced proliferative diseases. Such studies can in particular prove the synergism of the active ingredients of the COMBINATIONS OF THE INVENTION. The beneficial effects on proliferative diseases can be determined directly through the results of these studies or by changes in the study design which are known as such to a person skilled in the art. Such studies are, in particular, suitable to compare the effects of a monotherapy using the active ingredients and a COMBINATION OF THE INVENTION. Preferably, the combination partner (a) is administered with a fixed dose and the dose of the combination partner (b) is escalated until the Maximum Tolerated Dosage is reached. In a preferred embodiment of the study, each patient receives daily doses of the combination partner (a). The efficacy of the treatment can be determined in such studies, e.g., after 18 or 24 weeks by radiologic evaluation of the tumors every 6 weeks.

Alternatively, a placebo-controlled, double blind study can be used in order to prove the benefits of the COMBINATION OF THE INVENTION mentioned herein.

The COMBINATION OF THE INVENTION can also be applied in combination with surgical intervention, mild prolonged whole body hyperthermia and/or irradiation therapy.

The COMBINATION OF THE INVENTION can be a combined preparation or a pharmaceutical composition.

It is one objective of this invention to provide a pharmaceutical composition comprising a quantity, which is jointly therapeutically effective against a proliferative disease comprising the COMBINATION OF THE INVENTION. In this composition, the combination partners (a) and (b) can be administered together, one after the other or separately in one combined unit dosage form or in two separate unit dosage forms. The unit dosage form may also be a fixed combination.

The pharmaceutical compositions for separate administration of the combination partners (a) and (b) and for the administration in a fixed combination, i.e. single galenical compositions comprising at least two combination partners (a) and (b), according to the invention can be prepared in a manner known per se and are those suitable for enteral, such as oral or rectal, and parenteral administration to mammals (warm-blooded animals), including man, comprising a therapeutically effective amount of at least one pharmacologically active combination partner alone or in combination with one or more pharmaceutically acceptable carriers, especially suitable for enteral or parenteral application.

Novel pharmaceutical compositions contain, for example, from about 10% to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each of the combination partners of the COMBINATION OF THE INVENTION may be administered simultaneously or sequentially and in any order, and the components may be administered separately or as a fixed combination. For example, the method of delay of progression or treatment of a proliferative disease according to the invention may comprise (i) administration of the combination partner (a) in free or pharmaceutically acceptable salt form and (ii) administration of a combination partner (b) in free or pharmaceutically acceptable salt form, simultaneously or sequentially in any order, in jointly therapeutically effective amounts, preferably in synergistically effective amounts, e.g. in daily dosages corresponding to the amounts described herein. The individual combination partners of the COMBINATION OF THE INVENTION can be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimes of simultaneous or alternating treatment and the term “administering” is to be interpreted accordingly.

An example of sequential administration could be a first administration of N-{5-[4-(4-methyl-piperazino-methyl)-benzoylamido]-2-methylphenyl}-4-(3-pyridyl)-2-pyrimidine-amine until a resistance to the therapy is observed, followed by the administration of a hypusination inhibitor taken alone or in combination with Imatinib.

The effective dosage of each of the combination partners employed in the COMBINATION OF THE INVENTION may vary depending on the particular compound or pharmaceutical composition employed, the mode of administration, the condition being treated, the severity of the condition being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites. As many hypusination inhibitors have previously known therapeutic usefulness in other indications (e.g., antifungals, iron chelators) effective and safe dosage ranges may easily be determined by one of skill in the art and without undue experimentation with regard to the indication disclosed herein.

N-{5-[4-(4-methyl-piperazino-methyl)-benzoylamido]-2-methylphenyl}-4-(3-pyridyl)-2-pyrimidine-amine monomesylate, is preferably administered to a human in a dosage in the range of about 2.5 to 850 mg/day, more preferably 5 to 600 mg/day and most preferably 20 to 300 mg/day. Unless stated otherwise herein, the compound is preferably administered from one to four times per day, more preferably once daily.

Furthermore, the present invention pertains to the use of a COMBINATION OF THE INVENTION for the delay of progression or treatment of a proliferative disease and to the use of a COMBINATION OF THE INVENTION for the preparation of a medicament for the delay of progression or treatment of a proliferative disease.

Preferably, the proliferative disease is leukemia, Imatinib-resistant leukemia or tumors.

Moreover, the present invention provides a commercial package comprising a COMBINATION OF THE INVENTION, together with instructions for simultaneous, separate or sequential use thereof in the delay of progression or treatment of a proliferative disease.

The following Examples illustrate the invention described above, but are not, however, intended to limit the scope of the invention in any way. The beneficial effects of the COMBINATION OF THE INVENTION (i.e. good therapeutic margin, less side effects, synergistic therapeutic effect and other advantages mentioned herein), can also be determined by other test models known as such to the person skilled in the pertinent art. The synergistic therapeutic effect, may for example, be demonstrated in a clinical study or in a test procedure familiar to one of skill in the art.

It is contemplated that the invention described herein is not limited to the particular methodology, protocols, and reagents described as these may vary. It is also to be understood that the terminology used herein is for the purpose of describing particular embodiments only, and is not intended to limit the scope of the present invention in any way.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are now described. All publications mentioned herein are incorporated by reference for the purpose of describing and disclosing the materials and methodologies that are reported in the publication which might be used in connection with the invention.

In practicing the present invention, many conventional techniques in molecular and cellular biology are used. These techniques are well known and are explained in, for example, Current Protocols in Molecular Biology, Volumes I, II, and III, 1997 (F. M. Ausubel ed.); Sambrook et al., 1989, Molecular Cloning: A Laboratory Manual, Second Edition, Cold Spring Harbor Laboratory Press, Cold Spring Harbor, N.Y.

### EXAMPLES

### Materials and Methods

### Reagents

A stock solution of Imatinib (10 mg/ml) is prepared by dissolving the compound in DMSO/H₂O (1:1) and stored at −20° C. The final concentration of dimethyl sulfoxide in the media is less than 0.1%, and had no effect on the cell growth inhibition in the present study. Ciclopirox (Sigma, Taufkirchen, Germany) is freshly dissolved in PBS (10 mg/ml) for the in vitro experiments.

### Cell Culture Techniques

K562 cells were obtained from DSMZ (Bielefeld, Germany). HL-60 lines were kindly provided by Dr. Bühring (Tübingen, Germany). Both cell lines are cultured in RPMI 1640 medium (Gibco-BRL, Invitrogen, UK) containing 10% fetal calf serum (FCS) (Biochrom KG, Berlin, Germany). The cells are incubated at 37° C. in a humidified atmosphere with 5% CO₂ in air.

### Cell Lysis and Protein Solubilization

Protein samples are isolated from 10⁷ K562 cells which yielded 1000 μg of protein. Cells are lysed in sample buffer, followed by centrifugation at 12000 g for 5 minutes. The protein concentration in the supernatant is determined according to the method of Bradford (Bradford, M., Anal. Biochem. 72, 248 (1976)).

### Two-Dimensional (2D) Gel Electrophoresis

Isoelectric focusing is performed as previously described (Görg et al. Electrophoresis 21, 1037-1053 (2000)). Samples are applied to IPG strips (pH 4-7, 18 cm, Amersham Biosciences) by in gel rehydration. Following isoelectric focussing on Multiphor II (Pharmacia, Sweden), IPG strips are equilibrated for 2×15 min in 6 M urea, 4% SDS, 50 mM Tris-HCl, pH 8.8, containing either 1% DTT for the first or 4.8% iodoacteamide for the second period of equilibration. Strips are placed on vertical SDS-PAGE gels and overlayed with 0.6% agarose. SDS-PAGE is carried out in Amersham Biosciences isoDalt system using gels of 1.5 mm thickness and an acrylamide concentration of 15% T, 2.5% C. 2D gels are stained over-night with colloidal Coomassie, followed by destaining for 1 day.

### Mass Spectrometry

In-gel digestion is performed as previously described (Shevchenko et al., Proc. Natl. Acad. Sci. U.S. A 93, 14440-14445 (1996)) with minor modifications. The protein spots are excised from the gel, washed with Millipore-purified water and with 50% acetonitrile/water. After drying, trypsin (sequencing grade, Promega, Mannheim, Germany) is added to each sample. Tryptic protein fragments are extracted from the gel matrix with 5% formic acid and with 50% acetonitrile/5% formic acid. The extracts are pooled and concentrated in a speed vac concentrator. After purification with ZipTips (C18-ZipTip, Millipore, Bedford, Mass., USA), aliquots are deposited on a spot of alpha-cyano-4-hydroxycinnamic acid/nitrocellulose and analyzed with a Reflex III MALDI-TOF mass spectrometer (Bruker Daltonic, Bremen, Germany) equipped with an N₂ 337 nm laser. All measurements are performed in the positive-ion reflection mode at an accelerating voltage of 23 kV and delayed-pulsed ion extraction. Sequence verification of tryptic fragments is performed by nanoelectrospray tandem mass spectrometry on a hybrid quadrupole orthogonal acceleration time-of-flight mass spectrometer (QSTAR Pulsar i, Applied Biosystems/MDS Sciex, Foster City, Calif., USA) equipped with a nanoflow electrospray ionization source. Purified aliquots are loaded in a nanoelectrospray needle (BioMedical Instruments, Zoellnitz, Germany) and tandem mass spectra are obtained by collision-induced decay of selected precursor ions. The instrument is calibrated externally.

Database searches (NCBlnr, non-redundant protein database) are performed using the MASCOT software from Matrix Science (Perkins et al., Electrophoresis 20, 3551-3567 (1999)) with carboxymethylation of cysteine and methionine oxidations as variable modifications (probability value p<0.05).

### Western Blotting

For protein extraction, cells are homogenized in lysis buffer containing 50 mM Tris-HCl, pH 7.5, 150 mM NaCl, 1% NP-40, 0.25% Na-desoxycholate, 5 mM EDTA, 1 mM NaF, 25 mM Na₃VO₄ and 0.1 mM PMSF on ice. The lysates are left on ice for 10 min and cellular debris is pelleted at 14000 rpm for 20 min at 4° C. The supernatant is frozen at −80° C. The protein concentration of the lysate is determined with the BCA Protein Assay Kit (Pierce, Rockford, USA).

Proteins (20 μg) are separated by 12.5% SDS-PAGE and transferred onto nitrocellulose membranes using the Bio-Rad Transblot system. After blocking in TBS-Tween/5% w/v BSA for 1 h, membranes are incubated in primary antibody diluted in TBS-Tween/5% w/v BSA. Following primary antibody are used: Vinculin, RHO-GDI. After washing, membranes are incubated for 1 h either in HRP-conjugated rabbit anti-goat Ig (1/10000) or rabbit anti-mouse Ig (1/10000) diluted in TBS-T/5% w/v BSA. After washing, the enhanced chemiluminescence kit (Amersham Pharmacia Biotech UK Ldt.) is used to visualize the secondary antibody.

### MTT Assay

K652 and HL-60 cells are plated into 96-well flat-bottomed microtiter plates (Becton Dickinson, Heidelberg, Germany) at 1.5×10⁴ cells/well in 150 μl of their respective media. Cells are preincubated for 24 h before Imatinib (0 to 3 μM) or ciclopirox (0 to 81 μM) or the combination of both drugs are added at increasing concentrations.

All analyses are performed in triplicates. After 24 and 48 hours, the viable cells in each well are assayed for their ability to transform 3-(4,5-dimethylthiazol-2-yl)-2,5-diphenyltetrazolium bromide (MTT) into a purple formazan (Twentyman et al., Br. J. Haematol. 71, 19-24 (1989), Arnould et al., Anticancer Res. 10, 145-154 (1990)). Therefore, 10 μl of a 10 mg/ml MTT solution is added in each well. After an incubation period of 2 hours at 37° C., the purple formazan is released by adding lysis buffer (15% sodium dodecyl sulfate [SDS] in DMF/H₂O 1:1, pH 4.5) and shaking overnight in the dark. The absorbance of the samples is measured on an enzyme-linked immunosorbent assay (ELISA) plate reader (Dynatech MR7000) at 570 nm. The dose-effect relationship for Imatinib at the point of IC₅₀ is analyzed by the median-effect method (Chou et al, Eur. J. Biochem. 115, 207-216 (1981), Chou et al., Adv. Enzyme Regul. 22, 27-55 (1984)) using the Calcusyn Software (Biosoft, Cambridge, UK). The IC₅₀ is defined as the concentration of drug that produces 50% cell growth inhibition and corresponds to the affected fraction (Fₐ value) of 0.5.

### Apoptosis

K562 and HL-60 (2×10⁵ cells per well) are cultured in 24-well tissue plates under the conditions described above. After 24 h of pre-incubation, cells are incubated in 0.15 μM Imatinib and at increasing concentrations of ciclopirox (0 to 81 μM) and sampled at 24 to 48 hours before the fraction of apoptotic cells are measured by flow cytometry according to Nicoletti et al. (Nicoletti et al., J. Immunol. Methods 139, 271-279 (1991)).

Briefly, nuclei are prepared by lysing cells in a hypotonic lysis buffer (1% sodium citrate, 0.1% Trition X-100, 50 μg propidium iodide per ml) and subsequently analyzed by flow cytometry. Nuclei to the left of the 2N peak containing hypodiploid DNA are considered as apoptotic. Flow cytometric analysis are performed on a FACScalibur (Becton Dickinson) using CELLQUEST analysis software.

### Example 1

### Differential Protein Expression

The delineation of the intracellular signalling cascades induced by the Bcr-Abl tyrosine kinase represents a prerequisite of a better understanding of the biology of Philadelphia chromosome (Ph)-positive leukemias. In the present example the differential protein expression of the well-established Bcr-Abl positive K562 cell line upon treatment with Imatinib in vitro for 24 and 48 hours is determined.

Two-dimensional gel analysis of proteins from Bcr-Abl-positive K562 cells is performed to produce a protein profile from K562 cells incubated with and without 4 micromolar Imatinib for 24 hours. A total of 1000 μg protein are separated by 2-D gel electrophoresis using an IPG gel with pH a range of 4-7 (first dimension), 15% acrylamid gels (second dimension) and proteins are visualized with colloidal coomassie. Particular protein spots are chosen for further characterization by MALDI-MS and ESI-MS/MS because they are highly expressed in control (data not shown).

By comparative analysis of treated versus untreated cells one can detect nineteen differentially expressed proteins, seven of which are over-expressed under Imatinib treatment, whereas twelve are found to be down-regulated. Only candidate proteins that are reproducibly detected at both 24 and 48 hours in three independent experiments are considered significant in terms of differential expression.

### Example 2

### Identification of Proteins

Using a proteomics approach to analyze Imatinib-induced differential protein expression associated with Bcr-Abl signalling in K562 cells, proteins are found to be differentially regulated. Once identified they can be classified due to their known biological function.

Identification of candidate proteins is performed via peptide mass-fingerprinting and peptide sequencing using MASCOT search tool and NCBI nr database as described above.

Immunoblots of selected representative proteins are also performed in order to confirm the results of the 2-D gels. Cell extracts are prepared in lysis buffer, equal amounts of protein were separated on 12.5% polyacrylamid gels and transferred to nitrocellulose membranes. Detection of α-tubulin is used to ensure comparable protein content in all lanes (data not shown).

The results indicate that of the proteins analyzed and identified, seven could be linked to cell cycle regulation and proliferation control, seven are involved in the regulation of focal adhesion and cytoskeletal organization, two proteins play a role in nuclear import/export, two proteins are involved in amino acid/purine metabolism and the function of two other proteins is still unknown. One downregulated protein of particular interest, eIF5A, the only known eukaryotic protein that is activated by posttranslational hypusination, became the focus of further studies.

### Example 3

### eIF5A and Synergistic Effects of Imatinib and Ciclopirox

While the underlying mechanisms of action are poorly understood, it has been suggested that the activity of Interferon-alpha and Ara-C and other drugs that are currently being used for the treatment of CML may involve inhibition of hypusination of eIF5A.

Hypusination is induced stepwise by two mechanisms. In a first step, catalyzed by the enzyme deoxyhypusine-synthase, deoxyhypusine intermediates are formed by NAD-dependent transfer of 4-aminobutyl to lysine residues of the eIF5a precursor. The second step generates the active form of eIF5a and involves the hydroxylation of the side chain of the deoxyhypusine intermediates by a second enzyme called deoxyhypusine hydroxylase.

eIF5a seems to be essential for proliferation of cells, since disruption of hypusine synthesis leads to cell cycle arrest. The minor human isoform, eIF5a2, has been suspected to be an oncogene. It is speculated that eIF5a facilitates transport and/or translation of specific mRNAs. Thus, Bcr-Abl induced upregulation of eIF5a could potentially play a role in the increased cellular proliferation observed in Bcr-Abl positive leukemias. Similarly, inhibition of Bcr-Abl could exert its anti-proliferative effect via inhibition of eIF5a expression.

In order to test this hypothesis, we investigated whether additive or even synergistic effects could be detected by treating Bcr-Abl positive leukemia cells with hypusination inhibitors and Imatinib. Specifically, we analyzed potential synergistic effects between Imatinib and ciclopirox on Bcr-Abl positive K562 cells by measuring cellular cytotoxicity and apoptosis.

Using a tetrazolium-based MTT assay, we quantified growth inhibition in K562 cells after 24 h of exposure to ciclopirox or Imatinib alone as well as to a combination of both drugs in the K562 cells and also in Bcr-Abl-negative HL-60 cells. The cells were treated with ciclopirox or Imatinib at increasing concentrations as follows: K562 cells were treated with 0, 0.33, 1, 3, 9, 27, 81 μM ciclopirox and/or 0, 0.01, 0.037, 0.11, 0.33, 1.0, 3.0 μM Imatinib; HL-60 cells were treated with 0, 0.33, 1, 3, 9, 27, 81 μM ciclopirox and/or 0, 0.33, 1, 3, 9, 27, 81 μM Imatinib.

Whereas an anti-proliferative effect was detected with ciclopirox alone, data indicate that the combination of Imatinib and ciclopirox were significantly synergistic on cellular cytotoxicity in Bcr-Abl positive K562 cells. In contrast, no synergistic effect with Imatinib was observed when the Bcr-Abl negative myeloid leukemia cell line HL60 was treated with both drugs as Bcr-Abl-negative HL-60 cells were not affected by this combination. Results are representative of at least 3 independent experiments (data not shown).

Apoptosis was also measured after 24 h by flow cytometric evaluation of hypodiploid nuclei as described in above Methods. In these experiments, K562 and HL-60 cells were treated with ciclopirox at increasing concentration (0 to 81 pMor with 0.15 μM Imatinib and ciclopirox at increasing concentration (0 to 81 μM). Data indicate that Imatinib sensitizes Bcr-Abl-positive K562 cells but not Bcr-Abl negative HL-60 cells to ciclopirox-induced apoptosis. Results are representative of at least 3 independent experiments (data not shown).

Our findings support the central role of eIF5A for cell cycle control in Bcr-Abl-positive leukemias and points to this protein as being a potential new target for future therapies. Interestingly, among the substances known to inhibit hypusination, deferoxamine (iron overload agent) and ciclopirox (topically used anti-fungal) are clinically approved drugs with an acceptable toxicity profile. Thus, based on the results reported herein, it is contemplated that clinical treatment strategies combining hypusination inhibitors with/without Imatinib could be used to reduce the development of clinical resistance to Imatinib in Bcr-Abl positive leukemias, as well as other disease entities treated with Imatinib.

1: A method of treating a warm-blooded animal having a proliferative disease comprising administering to the animal a combination which comprises (a) N-{5-[4-(4-methyl-piperazino-methyl)-benzoylamido]-2-methylphenyl}-4-(3-pyridyl)-2-pyrimidine-amine and (b) at least one hypusination inhibitor, in a quantity which is jointly therapeutically I effective against a proliferative disease and in which the compounds can also be present in the form of their pharmaceutically acceptable salts.

2: The method according to claim 1, wherein the proliferative disease is leukemia or Imatinib-resistant leukemia.

3: A method of treating a warm-blooded animal having leukemia, particularly an Imatinib-resistant leukemia, comprising administering to the animal at least one hypusination inhibitor, in a quantity which is therapeutically effective against leukemia and in which the compounds can also be present in the form of their pharmaceutically acceptable salts.

4: A combination which comprises (a) N-{5-[4-(4-methyl-piperazino-methyl)-benzoylamido]-2-methylphenyl}-4-(3-pyridyl)-2-pyrimidine-amine and (b) at least one hypusination inhibitor, wherein the active ingredients are present in each case in free form or in the form of a pharmaceutically acceptable salt, and optionally at least one pharmaceutically acceptable carrier; for simultaneous, separate or sequential use.

5: Combination according to claim 4 wherein the compound (a) is used in the form of its monomethanesulfonate salt.

6: Combination according to claim 4, which is a combined preparation or a pharmaceutical composition.

7: A pharmaceutical composition comprising a quantity which is jointly therapeutically effective against a proliferative disease of a combination according to claim 4 and at least one pharmaceutically acceptable carrier.

8-12. (canceled)

13: The combination composition according to claim 4, in which the combination partners (a) and (b) are administered in synergistically effective amounts.

14: A commercial package comprising a combination according to claim 4, together with instructions for simultaneous, separate or sequential use thereof in the delay of progression or treatment of a proliferative disease.

15: The method according to claim 1, in wherein the hypusination inhibitor is selected from the group consisting of deferoxamine, ciclopirox, deoxyspergualin, deferiprone and GC-7.

16: The method, according to claim 1, in wherein the hypusination inhibitor is 4-[3,5-bis(2-hydroxyphenyl)-[1,2,4]triazol-1-yl]benzoic acid.

17: The method according to claim 1, in wherein the hypusination inhibitor is ciclopirox.
